(19) United States
(12) Patent Application Publication
(10) Pub. No.: US 2005/0226924 A1
(43) Pub. Date: Oct. 13, 2005

(54) Composition comprising itraconazole for oral administration

(76) Inventors: Kyu-Hyun Lee, Incheon (KR); Eun-Seok Park, Suwon (KR); Sang-Cheol Chi, Suwon (KR)

Correspondence Address: MUSERLIAN, LUCAS AND MERCANTI, LLP, 475 PARK AVENUE SOUTH, 15TH FLOOR, NEW YORK, NY, 10016, US

(21) Appl. No.: 10/902,713

(22) Filed: Jul. 29, 2004

(30) Foreign Application Priority Data
Apr. 13, 2004 (KR) 10-2004-0025490

(51) Int. Cl. A61K 31/496; A61K 9/20; A61K 9/14

(52) U.S. Cl. 424/464; 514/254.07

(57) ABSTRACT

The present invention relates to a composition comprising itraconazole for oral administration, more precisely, a composition for oral administration containing 1 part by weight itraconazole, 0.1-0.5 part by weight citric acid and 0.1-0.5 part by weight hydroxypropylmethylcellulose. The composition of the present invention has the advantages of mitigating discomfort of administration by reducing the amount of the additives used to make itraconazole water-soluble, lowering the production price by shortening processing time of spray-drying, high solubility and dissolution rate, excellent reproducibility and stability.

### FIELD OF THE INVENTION

The present invention relates to a composition comprising itraconazole for oral administration, more precisely, a composition for oral administration containing 1 part by weight itraconazole, 0.1-0.5 part by weight citric acid and 0.1-0.5 part by weight hydroxypropylmethylcellulose.

### BACKGROUND

Itraconazole [(±)-cis-4-[4-[4-[4-[[2-(2,4-dichlorophenyl)-2-(1H-1,2,4-triazole-1-yl-methyl)-1,3-dioxolane-4-yl]methoxy]phenyl]-1-piperazinyl]phenyl]-2,4-dihydro-2-(1-methylpropyl)-3H-1,2,4-triazole-3-one] is one of tricyclic azole compounds showing an excellent therapeutic effect on mycosis. Its molecular formula is C₃₅H₃₀Cl₂N₈O₄, and molecular weight is 705.649 g/mol. Itraconazole is powder having white or light yellow color. It is practically insoluble in water (less than 1 μg/ml), very slightly soluble in alcohol (300 μg/ml) but freely soluble in dichloromethane (239 mg/ml). Since itraconazole is a weak basic drug(pKa=3.7), it is ionized and completely soluble in low pH solution such as gastric juice. Bioavailability of itraconazole shows large variation among individuals, possibly due to food effect.

In the pharmaceutical formulation aspect, since itraconazole is a water-insoluble compound and has pH-dependent solubility, it is difficult to formulate itraconazole in an effective dosage form. So, the formulation research of itraconazole has been focused on increasing its solubility in water to improve bioavailability of the drug.

The formation of complex using cyclodextrin and its derivatives was described in WO No. 85/02767 and U.S. Pat. No. 4,764,604. However, the solubility and bioavailability of itraconazole could not be improved by the stated method. And it needs complicated process of various steps in actual production line.

The production of itraconazole in the form of a bead using a water-soluble polymer was described in WO No. 94/05263. The bead form of a three-layer structure, has been developed and introduced on market by Janssen Pharmaceutica Co. (Product name: Sporanox capsule). It was produced by the steps of coating a core, which is composed of pharmaceutically inactive sugar, dextrin and starch, with itraconazole and hydrophilic polymer and further coating it with another polymer such as polyethyleneglycol. However, the method has still problems in a manufacturing process. That is, cores tend to lump together because the core has small of 600-700 μm. Besides, the method requires a special machine and highly complicated manipulation.

The preparation of a solid dispersion using a water-soluble polymer and the drug using melt-extrusion method was also described in WO No. 97/44014. This method contributed to the increase of bioavailability of itraconazole, without being affected by food taken, which has been a problem of conventional products on the market. However, melt-extrusion of itraconazole should be performed at very high temperature of 245-265° C. and it is difficult to disperse the drug homogeneously in the polymer. Besides, a part of itraconazole might not be melted completely and, thus, might affect its dissolution or absorption, indicating that it is difficult to produce the itraconazole product of uniform propeties.

The preparation of eutectic mixture using organic acids and itraconazole was described in Korean Patent Laid-open No. 10-1999-1565 and the preparation of melting mixture using sugars and the drug was described in Korean Patent Laid-open No. 10-1999-51527. However, those methods also have a problem that they can increase solubility by preparing of solid dispersion only with at least equal amount of additives.

The preparation of a melting dispersion using phosphoric acid and itraconazole was described in Korean Patent Laid-open No. 10-2001-2590. In this case, phosphoric acid, a strong acid, is used to improve solubility and dissolution rate, so that it might injure the stomach when orally administered.

According to the previous descriptions mentioned above, previous methods have the limitation in the development of itraconazole into a dosage form, because they require large amount of additives, which makes the patient to have difficulties in swallowing and results in low reproducibility.

After all the efforts the present inventors have made to overcome the above problems, the present inventors have completed this invention by determining the best mixing ratio of itraconazole, citric acid and hydroxypropylmethylcellulose, in which itraconazole was formulated into water-soluble preparation which is easy to swallow and reproducible to produce.

### SUMMARY OF THE INVENTION

It is an object of this invention to provide a composition for oral administration containing itraconazole, citric acid and hydroxypropylmethylcellulose.

### DETAILED DESCRIPTION OF PREFERRED EMBODIMENTS

The present invention provides a composition for oral administration containing itraconazole, citric acid and hydroxypropylmethylcellulose.

Hereinafter, the present invention is described in detail.

The present invention provides a composition for oral administration containing itraconazole 1 part by weight, citric acid 0.1-0.5 part by weight and hydroxypropylmethylcellulose 0.1-0.5 part by weight.

When the contents of citric acid and hydroxypropylmethylcellulose in the composition of the present invention were less than 0.1 part by weight, solubility of itraconazole was not improved satisfactorily, resulting in still low dissolution rate and low bioavailability. When their contents were over 0.5 part by weight, hygroscopicity increased too fast to store the composition stably without color change even though the solubility of itraconazole was improved satisfactorily.

When the composition of the present invention included itraconazole 1 part by weight, citric acid 0.25 part by weight and hydroxypropylmethylcellulose 0.25 part by weight, solubility, dissolution rate and stability were all very excellent, indicating that it was the optimum condition.

A solid dispersion according to the present invention can be prepared by spray-drying method as follows.

First, a solution (8%(W/W)) is prepared by dissolving itraconazole, citric acid and hydroxypropylmethylcellulose in an organic solvent. The solution is dried in a spray-dryer or in a fluid-bed granulator, resulting in a solid dispersion.

Various organic solvents can be used as an organic solvent of the present invention. A mixed solvent of dichloromethane and ethanol is preferred and, in particular, a mixed solvent of dichloromethane and ethanol at the ratio of 6:4 (weight to weight ratio) is more preferred.

The operation condition for spray-drying using a spray dryer is as follows; inlet temperature is 50˜60° C., aspirator is −25 mbar, and air flow rate is 600˜800 Nl/h. The operation condition for spray-drying using a fluid-bed granulator is as follows; inlet temperature is 50˜60° C., outlet temperature is 30˜40° C., and granule temperature is 25˜35° C.

The solid dispersion of the present invention can be formulated into oral dosage forms by mixing with pharmaceutical-grade diluents, binders, disintegrants, lubricants, etc.

Starch, lactose, sugar, mannitol, sorbitol, glucose, microcrystalline cellulose, calcium phosphate dibasic, etc. can be used as a diluent in this invention.

Starch, gelatin, polyvinylpyrrolidone, gum arabic, cellulose derivatives such as methyl cellulose, ethyl cellulose, hydroxypropylmethylcellulose, sodium carboxymethylcellulose, etc. can be used as a binder in this invention.

Starch, starch derivatives like sodium starchglycolate, carboxymethylcellulose derivatives such as calcium carboxymethylcellulose and crosslinked carboxymethylcellulose, microcrystalline cellulose, crosslinked polyvinylpirrolidone, etc. can be used as a disintegrating agent in this invention.

Stearic acid and its alkaline metal salts or amine salts, colloidal silicon dioxide, silicates, talc., etc. can be used as a lubricant in this invention.

The solid dispersion according to the present invention can be formulated into various oral dosage forms such as tablets, powders, granules and capsules using conventional methods.

The weight of a composition of the present invention, regardless of the dosage form, is about 300 mg containing 100 mg itraconazole. Therefore, the present invention provides a formulation for easy administration, overcoming discomfort in administration of conventional products of big sizes and particularly helping patients or others who have difficulty in swallowing of the preparations.

### EXAMPLES

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1˜3

### Preparation of a Solid Dispersion Using Spray-drying Method

### Example 1

10 g of itraconazole, 1 g of citric acid and 1 g of hydroxypropylmethylcellulose were dissolved in a mixed solvent of dichloromethane and ethanol (6:4 weight to weight ratio), resulting in an 8% (w/w) solution. The solution was dried in a spray-dryer (Model; B-191, Buchi), resulting in a solid dispersion.

The conditions for the spray-drying were as follows; inlet temperature: 55° C., aspirator: −25 mbar, air flow rate: 650 Nl/h.

### Example 2

10 g of itraconazole, 2.5 g of citric acid and 2.5 g of hydroxypropylmethylcellulose were dissolved in a mixed solvent of dichloromethane and ethanol (6:4 weight to weight ratio), resulting in an 8% (w/w) solution. Then, the solid dispersion was prepared using same procedure as described in Example 1.

### Example 3

10 g of itraconazole, 5 g of citric acid and 5 g of hydroxypropylmethylcellulose were dissolved in a mixed solvent of dichloromethane and ethanol (6:4 weight to weight ratio), resulting in an 8% (w/w) solution. Then, the solid dispersion was prepared using same procedure as described in Example 1.

### Example 4

### Preparation of Solid Dispersion Granules Using a Fluid-bed Granulator

239 g of lactose, 18 g of polyvinylpirrolidone and 60 g of sodium starch glycolate were put in a fluid-bed granulator (Model; GPCG-1, Glatt). In the meantime, 200 g of itraconazole, 40 g of citric acid and 40 g of hydroxypropylmethylcellulose were dissolved in a mixed solvent of dichloromethane and ethanol (6:4 weight to weight ratio), resulting in an 8% (w/w) solution. The solution was sprayed into the fluid-bed granulator, resulting in solid dispersion granules.

The conditions for the spray-drying was as follows; inlet temperature was 55° C., outlet temperature was 35° C., and granule temperature was 30° C.

### Example 5˜6

### Preparation of Tablets

### Example 5

150 parts by weight of the solid dispersion prepared in the above Example 2, 118.5 parts by weight of lactose and 30 parts by weight of sodium crosschamelose were mixed together, to which purified water was added to prepare granules. The mixture was dried at 50° C. and was sieved into appropriate size. Then, 1.5 part by weight of magnesium stearate was added thereto. The total weight of a tablet containing itraconazole was 300 mg (100 mg as itraconazole)

### Example 6

1.5 part by weight of magnesium stearate was added to 298.5 parts by weight of the solid dispersion prepared in the above Example 4 and tabletted. The total weight of a tablet containing itraconazole was 300 mg (100 mg as itraconazole)

### Comparative Example 1

10 g of itraconazole, 0.5 g of citric acid and 0.5 g of hydroxypropylmethylcellulose were dissolved in a mixed solvent of dichloromethane and ethanol (6:4 weight to weight ratio), resulting in an 8% (w/w) solution and solid dispersion was prepared using the same method described in Example 1.

### Comparative Example 2

10 g of itraconazole, 10 g of citric acid and 10 g of hydroxypropylmethylcellulose were dissolved in a mixed solvent of dichloromethane and ethanol (6:4 weight to weight ratio), resulting in an 8% (w/w) solution and solid dispersion was prepared using the same method described in Example 1.

### Comparative Example 3

10 g of itraconazole, 30 g of citric acid and 30 g of hydroxypropylmethylcellulose were dissolved in a mixed solvent of dichloromethane and ethanol (6:4 weight to weight ratio), resulting in an 8% (w/w) solution and solid dispersion was prepared using the same method described in Example 1.

### Comparative Example 4

10 g of itraconazole and 2.5 g of citric acid were dissolved in a mixed solvent of dichloromethane and ethanol (6:4 weight to weight ratio), resulting in an 8% (w/w) solution and solid dispersion was prepared using the same method described in Example 1.

### Comparative Example 5

10 g of itraconazole and 2.5 g of hydroxypropylmethylcellulose were dissolved in a mixed solvent of dichloromethane and ethanol (6:4 weight to weight ratio), resulting in an 8% (w/w) solution and solid dispersion was prepared using the same method described in Example 1.

### Comparative Example 6

10 g of itraconazole was dissolved in a mixed solvent of dichloromethane and ethanol (6:4 weight to weight ratio), resulting in an 8% (w/w) solution and solid dispersion was prepared using the same method described in Example 1.

### Comparative Example 7

110 parts by weight of the solid dispersion prepared in the above Comparative Example 1, 158.5 parts by weight of lactose and 30 parts by weight of sodium crosschamelose were mixed together, to which purified water was added to prepare granules. The mixture was dried at 50° C. and was sieved into appropriate size. Then, 1.5 part by weight of magnesium stearate was added thereto. The total weight of a tablet containing itraconazole was 300 mg (100 mg as itraconazole).

### Experimental Example 1

### Solubility Test

Samples, equivalent to 30 mg of itraconazole, was added to 10 9 of the test solution. The test solution used was the dissolution medium (pH 1.2) for dissolution test of Korea Pharmacopeia. After the samples were ultrasonicated for 30 minutes, they were shaked at 25° C. for 24 hours. Then, they were centrifuged at 3000 rpm for 20 minutes. The obtained supernatant was filtered using a 0.45 μm membrane filter, followed by 10-fold dilution with methanol. The content of itraconazole was measured by HPLC.

The results are shown in Table 1.

TABLE 1

| Composition ingredient | | | | | |
|---|---|---|---|---|---|
| | | | Hydroxy | | |
| | | | propyl | | |
| | Itraco | Citric | methyl | Solubility | |
| | nazole | acid | cellulose | (μg/ml) | Property |
| Example 1 | 1 | 0.1 | 0.1 | 206.7 ± 8.7 | White |
| | | | | | powder |
| Example 2 | 1 | 0.25 | 0.25 | 215.7 ± 4.9 | White |
| | | | | | powder |
| Example 3 | 1 | 0.5 | 0.5 | 210.0 ± 2.6 | White |
| | | | | | powder |
| Example 4 | 1 | 0.20 | 0.20 | 212.7 ± 4.5 | White |
| | | | | | granule |
| Comparative | 1 | 0.05 | 0.05 | 76.4 ± 10.0 | White |
| Example 1 | | | | | powder |
| Comparative | 1 | 1 | 1 | 212.3 ± 14.6 | Gray |
| Example 2 | | | | | powder/ |
| | | | | | brown |
| | | | | | spotted |
| Comparative | 1 | 3 | 3 | 207.0 ± 28.2 | Dark brown |
| Example 3 | | | | | powder |
| Comparative | 1 | 0.25 | — | 53.8 ± 16.6 | White |
| Example 4 | | | | | powder |
| Comparative | 1 | — | 0.25 | 165.1 ± 4.5 | White |
| Example 5 | | | | | powder |
| Comparative | 1 | — | — | 27.2 ± 5.0 | White |
| Example 6 | | | | | powder |

| | | |
|---|---|---|
| Raw itraconazole not spray-dried | 1.5 ± 0.7 | White |
| | | powder |

* Solubility: Mean ± SD, n = 3
* Property: Results obtained after 3 month storage at room temperature

As shown in Table 1, when a composition according to the present invention included itraconazole 1 part by weight, citric acid 0.25 part by weight and hydroxypropylmethylcellulose 0.25 part by weight, it showed the optimum solubility (Example 2). On the contrary, when citric acid and hydroxypropylmethylcellulose were included less than 0.1 part by weight, solubility was much lower even with itraconazole 1 part by weight (Comparative Example 1) When citric acid and hydroxypropylmethylcellulose were included more than 1 part by weight each to itraconazole 1 part by weight, a composition showed high solubility but low stability accompanied with a color change (Comparative Example 2, Comparative Example 3).

In conclusion, a composition according to the present invention can have optimum conditions, for example the best solubility and the highest stability, only when itraconazole, citric acid, and hydroxypropylmethylcellulose are included at the ratio of 1:0.1˜0.5:0.1˜0.5 weight to weight.

### Experimental Example 2

### Dissolution Test

Six tablets were selected for dissolution test (Paddle method) of Korea Pharmacopeia. The medium used was the dissolution medium (pH 1.2). Temperature of the medium and rotation speed of the paddle were 37° C. and 100 rpm, respectively. At 45 minutes, the medium was taken and filtered using a 0.45 μm membrane filter. The content of itraconazole in the medium was determined by HPLC.

The results are shown in Table 2.

TABLE 2

| Sample | % dissolved |
|---|---|
| Example 5 | 92.7 ± 4.5% |
| Example 6 | 90.5 ± 4.2% |
| Comparative Example 7 | 43.6 ± 2.9% |

(Mean ± SD, n = 6)

As shown in Table 2, when a composition of the present invention included itraconazole 1 part by weight, citric acid 0.25 part by weight and hydroxypropylmethylcellulose 0.25 part by weight, it showed the highest percent dissolved. Thus, the above ratio for itraconazole, citric acid and hydroxypropylmethylcellulose was proved to be the optimized ratio for an effective composition of the present invention.

### INDUSTRIAL APPLICABILITY

The composition of the present invention provides great advantages of easy administration of the drug by reducing the amount of the additives used to make itraconazole water-soluble, lowering production price by shortening spray-drying processing time, improving solubility and dissolution rate, and providing excellent reproducibility and stability in storage.

1. A composition for oral administration containing itraconazole 1 part by weight, citric acid 0.1-0.5 part by weight and hydroxypropylmethylcellulose 0.1-0.5 part by weight.

2. The composition for oral administration as set forth in claim 1, wherein the composition contains itraconazole 1 part by weight, citric acid 0.25 part by weight and hydroxypropylmethylcellulose 0.25 part by weight.

3. A preparation method of the composition for oral administration of claim 1 comprising the following steps: dissolving itraconazole 1 part by weight, citric acid 0.1-0.5 part by weight and hydroxypropylmethylcellulose 0.1-0.5 part by weight in an organic solvent; and preparing a solid dispersion by spray-drying the same.

4. The preparation method as set forth in claim 3, wherein the solution is dried using a spray-dryer or a fluid-bed granulator.
